(19) United States
(12) Patent Application Publication
(10) Pub. No.: US 2015/0289846 A1
(43) Pub. Date: Oct. 15, 2015

(54) ULTRASONIC IMAGING APPARATUS AND METHOD FOR CONTROLLING ULTRASONIC IMAGING APPARATUS

(71) Applicant: SAMSUNG ELECTRONICS CO., LTD., Suwon-si (KR)

(72) Inventors: Sungchan PARK, Suwon-si (KR); Jooyoung KANG, Yongin-si (KR); Kyuhong KIM, Seoul (KR); Jungho KIM, Yongin-si (KR); Suhyun PARK, Hwaseong-si (KR)

(73) Assignee: SAMSUNG ELECTRONICS CO., LTD., Suwon-si (KR)

(21) Appl. No.: 14/684,770

(22) Filed: Apr. 13, 2015

(30) Foreign Application Priority Data
Apr. 14, 2014 (KR) 10-2014-0044452

(51) Int. Cl. A61B 8/08; A61B 8/00; A61B 8/14

(52) U.S. Cl. CPC A61B 8/5207; A61B 8/14; A61B 8/4488; A61B 8/461; A61B 8/54

(57) ABSTRACT

An ultrasonic imaging apparatus includes: an ultrasound probe configured to transmit ultrasonic waves to a target region of an object in a plurality of directions, and to receive vibration waves generated from the object; and an image processor configured to generate image signals in the plurality of directions based on the vibration waves generated according to transmission of the ultrasonic waves in the plurality of directions, and to combine the image signals in the plurality of directions, wherein the ultrasound probe includes ultrasound elements configured to respectively generate ultrasonic waves of different frequencies, the ultrasonic waves intersecting each other in the target region of the object.

### CROSS-REFERENCE TO RELATED APPLICATION

This application claims priority from Korean Patent Application No. 10-2014-0044452, filed on Apr. 14, 2014, in the Korean Intellectual Property Office, the disclosure of which is incorporated herein by reference in its entirety.

### BACKGROUND

1. Field

Apparatuses and methods consistent with exemplary embodiments relate to an ultrasonic imaging apparatus and a method of controlling the ultrasonic imaging apparatus.

2. Description of the Related Art

Imaging apparatuses for acquiring images of an object include a radiography apparatus, a computed tomography (CT) scanner, a magnetic resonance imaging (MRI) apparatus, and an ultrasonic imaging apparatus. The ultrasonic imaging apparatus, as compared with other imaging apparatuses, has a lower price and higher safety since the patients do not need to be exposed to radiation or to noise. Accordingly, the ultrasonic imaging apparatus is widely used in various fields, such as a medical field, a security field, etc.

The ultrasonic imaging apparatus uses ultrasonic waves to acquire images of an object such as a human body. The ultrasonic imaging apparatus may produce echo ultrasonic waves transferred from the object as an ultrasound image by irradiating ultrasonic waves to a target region inside the object and receiving ultrasonic waves reflected from the target region, thereby acquiring an image of the object. In detail, the ultrasonic imaging apparatus may collect echo ultrasonic waves using an ultrasound probe, convert the echo ultrasonic waves into electrical signals, and produce an ultrasound image corresponding to the echo ultrasonic waves based on the electrical signals. More specifically, the ultrasonic imaging apparatus may perform beamforming on the electrical signals, and produce an ultrasound image based on the beamformed signals. The ultrasound image may be displayed to a user, for example, a doctor or a patient through a display device such as a monitor installed in or connected to the ultrasonic imaging apparatus through a wired and/or wireless communication network.

### SUMMARY

provide an ultrasonic imaging apparatus for quickly acquiring an ultrasound image, and a method of controlling the ultrasonic imaging apparatus.

One or more exemplary embodiments also provide an ultrasonic imaging apparatus for quickly acquiring an ultrasound image with improved resolution, and a method of controlling the ultrasonic imaging apparatus.

One or more exemplary embodiments further provide an ultrasonic imaging apparatus for acquiring an ultrasound image without using a hydrophone as well as reducing a time consumed to collect ultrasonic waves when the ultrasound image is acquired using vibroacoustography, and a method of controlling the ultrasonic imaging apparatus.

One or more exemplary embodiments still further provide a method for preventing resolution deterioration in acquiring an ultrasound image that occurs due to performing transmission focusing with high resolution whereas reception focusing cannot be performed with high resolution in vibroacoustography.

In accordance with an aspect of an exemplary embodiment, an ultrasonic imaging apparatus includes: an ultrasound probe configured to irradiate ultrasonic waves to at least one target region of an object in a plurality of directions, and to receive vibration waves generated from the object; and an image processor configured to generate a plurality of image signals in the plurality of directions for the object, based on a plurality of vibration waves generated according to irradiation of ultrasonic waves in the plurality of directions, and to combine the plurality of image signals in the plurality of directions, wherein the ultrasound probe includes a plurality of ultrasound elements configured to generate ultrasonic waves of different frequencies intersecting at least one target region of the object.

In accordance with an aspect of another exemplary embodiment, an ultrasonic imaging apparatus includes: an ultrasound probe configured to irradiate ultrasonic waves of different frequencies intersecting at least one target region inside an object, and receiving vibration waves generated from the object according to interference of the ultrasonic waves of the different frequencies; and an image processor configured to produce an image for the object based on the received vibration waves, wherein the image processor produces a plurality of images of a plurality of directions for the object based on a plurality of vibration waves generated according to irradiation of ultrasonic waves in the plurality of directions, and combines the plurality of image signals of the plurality of directions to produce a combined image signal.

### BRIEF DESCRIPTION OF THE DRAWINGS

The above and/or other aspects will become more apparent by describing certain exemplary embodiments with reference to the accompanying drawings, in which:

FIG. 1 is a view for describing an ultrasonic imaging apparatus according to an exemplary embodiment;

FIG. 2 is a block diagram illustrating a configuration of an ultrasonic imaging apparatus according to an exemplary embodiment;

FIG. 3 is a block diagram illustrating a configuration of an ultrasound probe according to an exemplary embodiment;

FIG. 4 is a view for describing an operation of an ultrasound generator, according to an exemplary embodiment;

FIG. 5 is a view showing examples of waveforms of ultrasonic waves and interference waves at a target region of an object;

FIGS. 6, 7, and 8 are views for describing irradiation of ultrasonic waves of different frequencies according to exemplary embodiments;

FIG. 9 is a view for describing vibration waves generated at a target region of an object according to an exemplary embodiment;

FIG. 10 is a block diagram illustrating a configuration of an ultrasound receiver according to an exemplary embodiment;

FIG. 11 is a perspective view of an ultrasound receiver according to an exemplary embodiment;

FIG. 12 is a view for describing an ultrasound receiver according to an exemplary embodiment;

FIGS. 13 and 14 are views for describing an ultrasound probe according to exemplary embodiments;

FIG. 15 is a block diagram illustrating a configuration of an image processor according to an exemplary embodiment;

FIGS. 16A, 16B, and 16C are views for describing a method of combining a plurality of images according to an exemplary embodiment;

FIGS. 17A, 17B, and 17C are views for describing a method of combining a plurality of images according to an exemplary embodiment;

FIG. 18 is a view for describing a method of combining a plurality of images according to an exemplary embodiment;

FIG. 19 is a flowchart illustrating a control method of an ultrasonic imaging apparatus, according to an exemplary embodiment; and

FIG. 20 is a flowchart illustrating a control method of an ultrasonic imaging apparatus, according to another exemplary embodiment.

### DETAILED DESCRIPTION

Certain exemplary embodiments are described in greater detail below with reference to the accompanying drawings, wherein like reference numerals refer to like elements throughout.

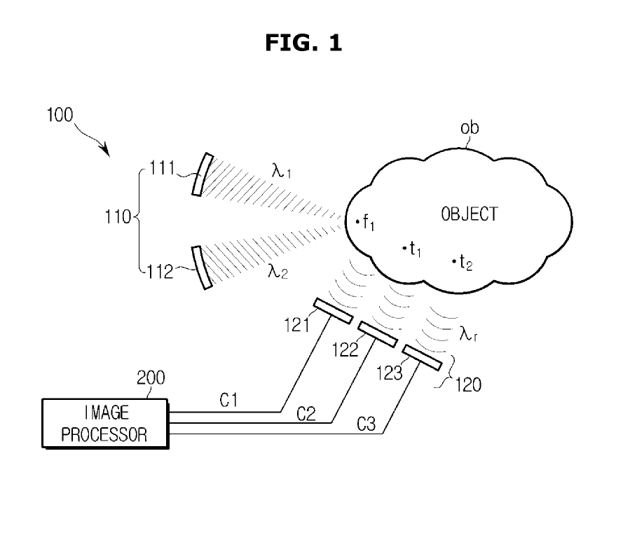

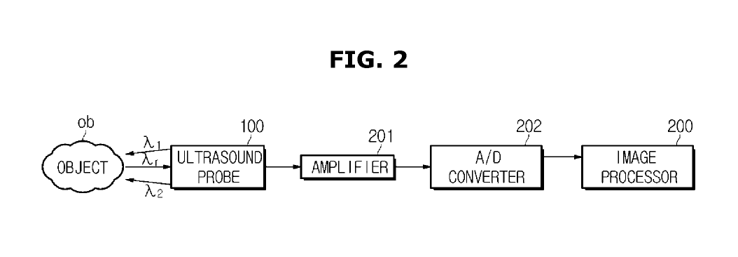

FIG. 1 is a view for describing an ultrasonic imaging apparatus according to an exemplary embodiment, and FIG. 2 is a block diagram illustrating a configuration of an ultrasonic imaging apparatus according to an exemplary embodiment.

Referring to FIGS. 1 and 2, an ultrasonic imaging apparatus may include an ultrasound probe 100 to collect information about the inside of an object ob, and an image processor 200 to produce a predetermined image based on information collected by the ultrasound probe 100.

The ultrasound probe 100 may irradiate a plurality of ultrasonic waves of different frequencies λ₁ and λ₂ to a target region f₁ inside the object ob, and receive vibration waves transferred from at least one of vibrating regions t₁ and t₂ inside the object ob. The target region f₁ may be a single region or a plurality of regions. Also, the vibrating regions t₁ and t₂ may be a single region or a plurality of regions.

The vibration waves received by the ultrasound probe 100 may be converted into electrical signals, and the electrical signals may be transferred to the image processor 200. The electrical signals may be electrical signals of a plurality of channels C1, C2, and C3. The image processor 200 may produce a plurality of images based on the electrical signals, and combine the plurality of images to produce a combined image.

According to an exemplary embodiment, the electrical signals output from the ultrasound probe 100 may be amplified by an amplifier 201 before the electrical signals are transferred to the image processor 200. Also, analog electrical signals output from the ultrasound probe 100 may be converted into digital electrical signals by an analog-digital (A/D) converter 202, and transferred to the image processor 200.

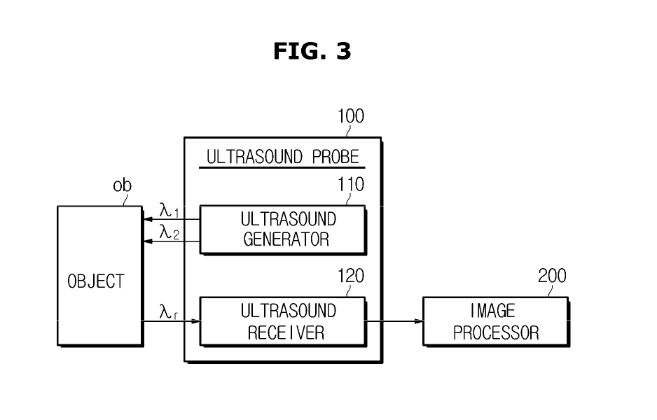

Hereinafter, the ultrasound probe 100 will be described in more detail. FIG. 3 is a block diagram illustrating a configuration of the ultrasound probe 100 according to an exemplary embodiment. Referring to FIGS. 1 to 3, the ultrasound probe 100 may include an ultrasound generator 110 to generate a plurality of ultrasonic waves of different frequencies λ₁ and λ₂, and an ultrasound receiver 120 to receive vibration waves having a frequency λᵣ that is reflected from or generated by the vibrating regions t₁ and t₂ of the object ob. The ultrasonic waves of different frequencies λ₁ and λ₂ generated by the ultrasound probe 100 may be irradiated to the target region f₁ inside the object ob. In FIG. 1, the ultrasound generator 110 includes a first and a second ultrasound generating elements 111, 112 to generate ultrasonic waves of different frequencies λ₁ and λ₂, respectively, and the ultrasound receiver 120 includes a first, a second, and a third ultrasound receiving elements 121, 122, 123 to receive vibration waves having a frequency λᵣ from the object ob and convert the received vibration waves into electrical signals of the plurality of channels C1, C2, and C3, respectively. However, exemplary embodiments are not limited thereto and the ultrasound generator 110 and the ultrasound receiver 120 may have any number of ultrasound generating elements and ultrasound receiving elements, respectively.

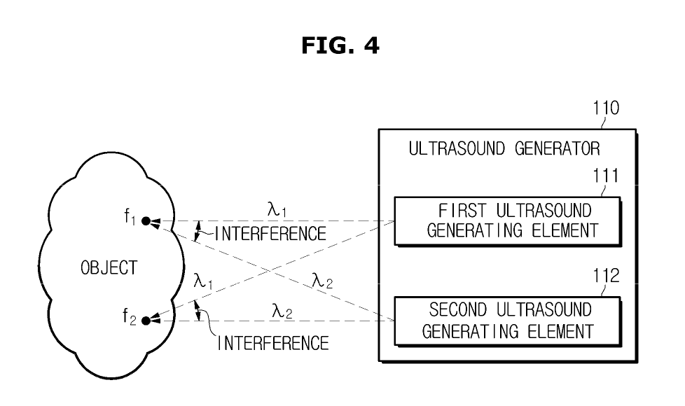

Hereinafter, the ultrasound generator 110 will be described. FIG. 4 is a view for describing an operation of the ultrasound generator 110, according to an exemplary embodiment.

The ultrasound generator 110 may include, as shown in FIG. 4, a plurality of ultrasound generating elements, for example, a first ultrasound generating element 111 and a second ultrasound generating element 112 to generate ultrasonic waves of different frequencies λ₁ and λ₂. However, the ultrasound generator 110 may include three or more ultrasound generating elements. The first and second ultrasound generating elements 111 and 112 may generate ultrasonic waves of different frequencies λ₁ and λ₂, independently or dependently. However, the ultrasound generator 110 may generate ultrasonic waves of three or more different frequencies. The ultrasonic waves of different frequencies λ₁ and λ₂ may be irradiated to target regions f₁ and f₂, respectively.

According to an exemplary embodiment, the ultrasound generator 110 may irradiate ultrasonic waves of different frequencies λ₁ and λ₂ using one of a plurality of target regions inside an object as a focal point. According to another exemplary embodiment, the ultrasound generator 110 may irradiate ultrasonic waves of different frequencies λ₁ and λ₂ using a plurality of target regions inside an object as focal points.

A plurality of ultrasonic waves of different frequencies λ₁ and λ₂ respectively generated by the ultrasound generating elements 111 and 112 of the ultrasound generator 110, for example, first ultrasonic waves of a first frequency λ₁ and second ultrasonic waves of a second frequency λ₂ may arrive at the same target region, for example, a first target region f₁ at substantially the same time or with a predetermined time difference. If the ultrasonic waves of different frequencies λ₁ and λ₂ arrive at the first target region f₁, materials of the first target region f₁ may be subject to a radiation force, e.g., an acoustic radiation force, according to the ultrasonic waves of different frequencies λ₁ and λ₂ to vibrate at a predetermined frequency. The vibration of the materials of the first target region f₁ results in generation of vibration waves.

More specifically, the ultrasonic waves of different frequencies λ₁ and λ₂ that arrived at the first target region f₁ may intersect each other, and the ultrasonic waves of different frequencies λ₁ and λ₂ intersecting each other may interfere so that the first target region f₁ may be influenced by the results of the interference of the ultrasonic waves of different frequencies λ₁ and λ₂.

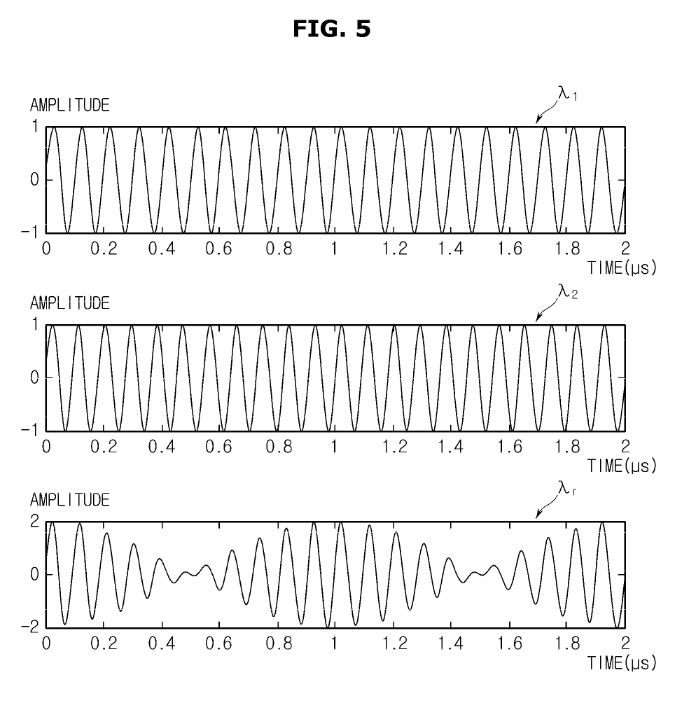

Referring to FIG. 4, the first ultrasound generating element 111 of the ultrasound generator 110 generates the first ultrasonic waves of the first frequency λ₁ and the second ultrasound generating element 112 generates the second ultrasonic waves of the second frequency λ₂, and the first and second ultrasonic waves may be irradiated to the same target regions f₁ and f₂. The first and second ultrasonic waves may intersect each other at or around the target regions f₁ and f₂. If the first and second ultrasonic waves intersect each other, the first and second ultrasonic waves may interfere to generate interference waves having an interference frequency λᵣ as shown in FIG. 5. In FIG. 5, examples of the first ultrasonic wave of the first frequency λ₁, the second ultrasonic waves of the second frequency λ₂, and the interference waves of the interference frequency λᵣ generated by interference between the first ultrasonic waves of the first frequency λ₁ and the second ultrasonic waves of the second frequency λ₂ are shown.

The ultrasonic waves of the different frequencies λ₁ and λ₂ intersecting each other may apply vibration of a predetermined frequency, that is, vibration of the interference frequency λᵣ to the materials of the first target region f₁. According to the vibration of the interference frequency λᵣ that is applied to the materials, the materials of the first target region f₁ may vibrate at a predetermined frequency. In this case, the vibration frequency of the materials may depend on the different frequencies λ₁ and λ₂ of the ultrasonic waves. In detail, the vibration frequency of the materials may depend on a frequency of interference waves, that is, an interference frequency λᵣ, as shown in FIG. 5. In other words, ultrasonic waves of different frequencies λ₁ and λ₂ intersect each other at the target region f₁ to apply vibration according to the interference frequency λᵣ to the materials of the target region f₁, and the materials to which the vibration is applied may vibrate according to the applied vibration.

According to an exemplary embodiment, as shown in FIG. 4, the ultrasound generator 110 may irradiate ultrasonic waves of two different frequencies λ₁ and λ₂, for example, the first ultrasonic waves of the first frequency λ₁ and the second ultrasonic waves of the second frequency λ₂ so that the first ultrasonic waves and the second ultrasonic waves interfere with each other to vibrate materials of the target region. According to another exemplary embodiment, the ultrasound generator 110 may irradiate ultrasonic waves of three or more different frequencies. Likewise, the ultrasonic waves of three different frequencies may intersect each other at the first target region f₁ and interfere with each other so that materials of the first target region f₁ vibrate. In other words, ultrasonic waves of three different frequencies may interfere with each other to produce interference waves.

The interference waves that are applied to the materials of the first target region f₁ can be obtained by using the following Equations (1) to (3), below.

Equation (1) expresses the first ultrasonic waves of the first frequency λ₁, and Equation (2) expresses the second ultrasonic waves of the second frequency λ₂.

Ψ₁=A sin(2πf₁t),and (1)

Ψ₂=A sin(2πf₂t), (2)

where ψ₁ represents the first ultrasonic waves, ψ₂ represents the second ultrasonic waves, f₁ and f₂ represent the first frequency λ₁ and the second frequency λ₂, respectively, t represents time, and A is a constant. Accordingly, vibration of the first target region f₁ caused by interference of the first and second ultrasonic waves can be expressed as Equation (3), below.

$$Ψ = {Ψ}_{1} + {Ψ}_{2} = 2 Acos ( 2 π \frac{{f}_{1} - {f}_{2}}{2} t ) sin ( 2 π \frac{{f}_{1} + {f}_{2}}{2} t ) , \qquad ( 3 )$$

where ψ represents the resultant waves appearing when the first and second ultrasonic waves interfere at the first target region f₁. That is, ψ means interference waves that apply vibration to the materials of the first target region f₁. The frequency and amplitude of the interference waves that are applied to the materials of the first target region f₁ may depend on the frequencies and amplitudes of the first and second ultrasonic waves respectively generated by the first ultrasound generating element 111 and the second ultrasound generating element 112. As seen in Equations (1) to (3), the frequency and amplitude of the interference waves may be different from the amplitude and/or frequency of the first ultrasonic waves or the amplitude and/or frequency of the second ultrasonic waves.

If the materials of the first target region f₁ vibrate according to vibration applied to the materials thereof, predetermined vibration waves may be generated from the materials of the first target region f₁. The vibration waves generated by the first target region f₁ may be radiated in all directions. The frequency of the vibration waves may depend on the vibration frequency of the materials of the first target region f₁. The generated vibration waves may be received by the ultrasound receiver 120 of the ultrasound probe 100.

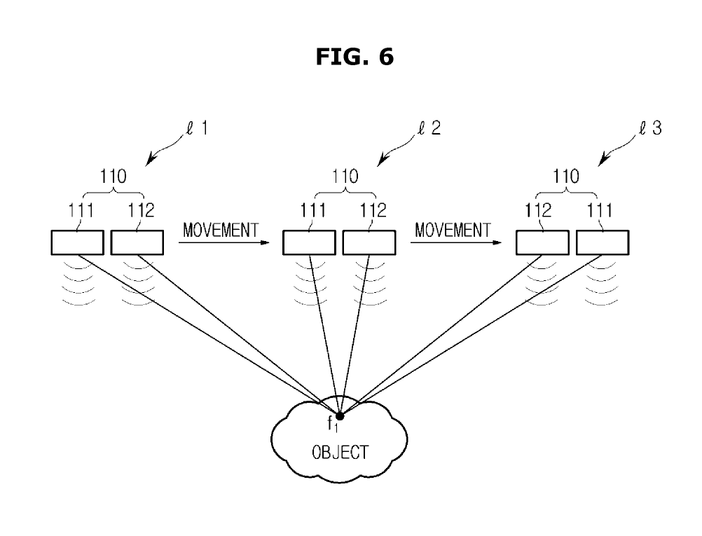

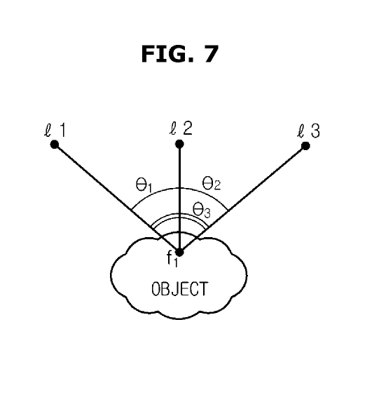

FIGS. 6 and 7 are views for describing irradiation of ultrasonic waves of different frequencies.

According to an exemplary embodiment, the ultrasound probe 100, or the ultrasound generator 110 of the ultrasound probe 100 may irradiate ultrasound waves of predetermined frequencies at a plurality of different locations I1 to I3, as shown in FIG. 6.

As shown in FIG. 6, the first and second ultrasound generating elements 111 and 112 of the ultrasound generator 110 may generate ultrasonic waves of different frequencies at a first location I1. The ultrasonic waves of different frequencies may be irradiated to the same target region f₁. As described above, the ultrasonic waves of different frequencies may intersect with each other at the target region f₁, and materials of the target region f₁ at which the ultrasonic waves of different frequencies intersect with each other may vibrate at a specific frequency due to interference of the ultrasonic waves of different frequencies to generate vibration waves. The generated vibration waves may be received by the ultrasound receiver 120 of the ultrasound probe 100.

The first and second ultrasound generating elements 111 and 112 may irradiate ultrasonic waves at substantially the same time or at different times. By delaying a timing at which any one of the ultrasound generating elements 111 and 112 generates ultrasonic waves by a predetermined time period, the respective ultrasound generating elements 111 and 112 can irradiate ultrasonic waves at different times. The ultrasound generating elements 111 and 112 may irradiate ultrasonic waves of different frequencies several times at the first location I1. When the ultrasound generating elements 111 and 112 irradiate ultrasonic waves, a plurality of vibration waves may be generated at the target region f₁, and the generated vibration waves may be received by the ultrasound receiver 120 of the ultrasound probe 100.

According to an exemplary embodiment, as shown in FIG. 6, after the ultrasound generator 110 irradiates ultrasonic waves or after the ultrasound receiver 120 collects ultrasound waves, the ultrasound probe 100 or the ultrasound generator 110 of the ultrasound probe 100 may move to a second location I2. For example, the ultrasound generator 110 may move in a direction that is substantially perpendicular to or substantially parallel to a depth direction axis. The ultrasound probe 100 or the ultrasound generator 110 may be moved by a user or by movement assistance means, such as a robot arm, a wheel, a rail, or the like.

After the ultrasound probe 100 or the ultrasound generator 110 of the ultrasound probe 100 moves to the second location I2, the ultrasound generating elements 111 and 112 of the ultrasound generator 110 may generate ultrasonic waves of different frequencies at the second location I2. The ultrasound generator 110 may irradiate the ultrasonic waves of different frequencies to the target region f₁, thereby irradiating ultrasonic waves to the target region f₁ in a direction that is different from the direction in which ultrasonic waves have been irradiated at the first location I1.

As shown in FIG. 6, the target region f₁ to which ultrasonic waves are irradiated at the second location I2 may be substantially the same as the target region to which ultrasonic waves have been irradiated at the first location I1. According to another exemplary embodiment, the target region f₁ to which ultrasonic waves are irradiated at the second location I2 may be different from the target region to which ultrasonic waves have been irradiated at the first location I1. Also, the frequency of the ultrasonic waves irradiated at the second location I2 may be the same as or different from the frequency of the ultrasonic waves irradiated at the first location I1.

An angle between an irradiation direction of ultrasonic waves of different frequencies irradiated at the second location I2 to the target region f₁ and an irradiation direction of ultrasonic waves of different frequencies irradiated at the first location I1 to the target region f₁ may be given as a first angle θ₁ as shown in FIG. 7. According to an exemplary embodiment, the first angle θ₁ may be a predetermined angle between 0 degree and 180 degrees. For example, the first angle θ₁ may be 45 degrees.

If ultrasonic waves of different frequencies are irradiated at the second location I2, materials of the target region f₁ may vibrate at a predetermined frequency due to interference of the ultrasonic waves of different frequencies to generate vibration waves. The generated vibration waves may be collected by the ultrasound receiver 120 of the ultrasound probe 100.

As described above, the ultrasonic waves of different frequencies may be irradiated several times at the second location I2, and vibration waves generated when the ultrasonic waves of different frequencies are irradiated may be collected.

The ultrasound probe 100 or the ultrasound generator 110 of the ultrasound probe 100 may move from the second location I2 to the third location I3. As described above, the ultrasound probe 100 or the ultrasound generator 110 may be moved by a user or by movement assistant means. If the ultrasound probe 100 or the ultrasound generator 110 are moved to the third location I3, the respective ultrasound generating elements 111 and 112 of the ultrasound generator 110 may generate ultrasonic waves of different frequencies at the third location I3, and irradiate the ultrasonic waves of different frequencies to the target region f₁ that is the same as or different from the region to which ultrasonic waves have been irradiated at the first location I1 or at the second location I2.

The target region f₁ to which the ultrasonic waves of different frequencies are irradiated at the third location I3 may be substantially the same as or different from the target region to which ultrasonic waves of different frequencies have been irradiated at the first location I1 or at the second location I2. Also, the frequency of the ultrasonic waves irradiated at the third location I3 may be the same as or different from the frequency of ultrasonic waves irradiated at the first location I1 or at the second location I2.

An angle between the irradiation direction of the ultrasonic waves of different frequencies irradiated at the third location I3 to the target region f₁ and the irradiation direction of the ultrasonic waves of different frequencies irradiated at the second location I2 to the target region f₁ may be given as a second angle θ₂, as shown in FIG. 7. Also, an angle between the irradiation direction of the ultrasonic waves of different frequencies irradiated at the third location I3 to the target region f₁ and the irradiation direction of the ultrasonic waves of different frequencies irradiated at the first location I1 to the target region f₁ may be given as a third angle θ₃, as shown in FIG. 7. The second angle θ₂ may be a predetermined angle between 0 degree and 180 degrees. For example, the second angle θ₂ may be 45 degrees. Meanwhile, the third angle θ₃ may be a sum of the first angle θ₁ and the second angle θ₂. In the case where the first and second angles θ₁ and θ₂ are each 45 degrees, the third angle θ₃ may be 90 degrees. In other words, a line segment connecting the target region f₁ to the first location I1 may be substantially perpendicular to a line segment connecting the target region f₁ to the third location I3.

As described above, the ultrasound receiver 120 of the ultrasound probe 100 may collect predetermined vibration waves generated from the target region f₁ after the ultrasonic waves of different frequencies are irradiated at the third location I3. The irradiation of ultrasonic waves and the collection of vibration waves may be performed several times.

Also, according to another exemplary embodiment, by adjusting irradiation timings of ultrasonic waves between a plurality of ultrasound generators 110 of the ultrasound probe 100, substantially the same effect as when the ultrasound generator 110 is moved as described above can be obtained. For example, by causing a plurality of ultrasound generators 110 arranged at different locations to generate ultrasonic waves at different times with predetermined time intervals, substantially the same effect as when the ultrasound generator 110 is moved to different locations can be obtained. In this case, the individual ultrasound generators 110 may irradiate ultrasonic waves at different times.

More specifically, by causing the ultrasound generator 110 arranged at a predetermined location (for example, the first location I1) among the plurality of ultrasound generators 110 respectively arranged at the first location I1 to the third location I3 to irradiate ultrasonic waves while causing the remaining ultrasound generators 110 arranged at the second and third locations I2 and I3 to stop irradiating ultrasonic waves, and then causing the ultrasound generator 110 arranged at another location (for example, the second location I2) to irradiate ultrasonic waves after a predetermined time period elapses while causing the remaining ultrasound generators 110 arranged at the first and third locations I1 and I3 to stop irradiating ultrasonic waves, substantially the same effect as when the ultrasound generator 110 is moved to irradiate at different locations may be obtained.

In this case, by adjusting a delayed irradiation time of ultrasonic waves of different frequencies that are irradiated at the second location I2 and an irradiation time of ultrasonic waves of different frequencies that are irradiated at the first location I1, it is possible to correctly focus the irradiated ultrasonic waves to the target region f₁.

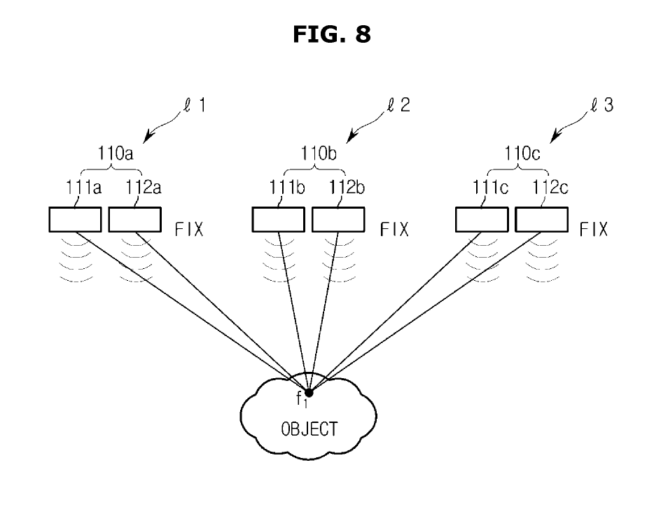

FIG. 8 is a view for describing irradiation of ultrasonic waves of different frequencies, according to another exemplary embodiment. As shown in FIG. 8, an ultrasonic imaging apparatus may include a plurality of ultrasound generators 110a, 110b, and 110c. The individual ultrasound generators 110a to 110c may include a plurality of ultrasound generating elements 111a, 112a, 111b, 112b, 111c, and 112c that can generate ultrasonic waves of different frequencies. The ultrasound generators 110a to 110c may be fixed at predetermined locations, for example, at first to third locations I1 to I3 to irradiate interfering ultrasonic waves to the target region f₁ of the object in different directions. According to an exemplary embodiment, the plurality of ultrasound generators 110a to 110c may irradiate interfering ultrasonic waves to the target region f₁ according to a predetermined pattern, for example, in a sequential manner.

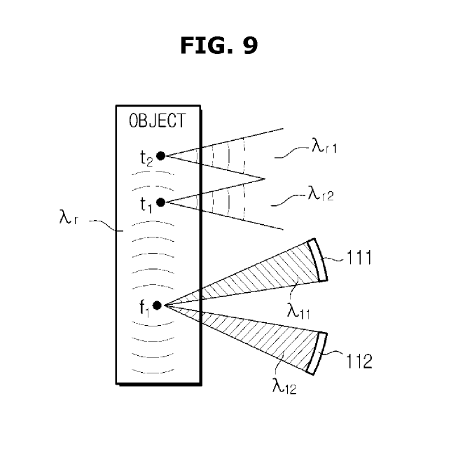

FIG. 9 is a view for describing vibration waves generated at a target region of an object according to an exemplary embodiment. When interfering ultrasonic waves of different frequencies λ₁₁ and λ₁₂ arrive at materials of a predetermined target region f₁, the ultrasonic waves of different frequencies λ₁₁ and λ₁₂ may interfere with each other so that the materials of the predetermined target region f₁ may vibrate at a specific frequency according to the results of the interference. The materials of the predetermined target region f₁ may generate vibration waves of a predetermined frequency λᵣ while vibrating. The vibration waves of the predetermined frequency λᵣ that is generated from the predetermined target region f₁ may be collected by the ultrasound probe 100.

When the vibration waves generated from the predetermined target region f₁ are radiated, the radiated vibration waves of the predetermined frequency λᵣ may transfer vibration to other materials around the materials of the target region f₁, as shown in FIG. 9. Accordingly, materials of other regions (for example, a first vibrating region t₁ and a second vibrating region t₂) other than the materials of the predetermined target region f₁ may also vibrate according to the vibration waves. In this case, the materials of the first vibrating region t₁ and the second vibrating region t₂ may also generate vibration waves of predetermined frequencies λᵣ₁ and λᵣ₂, similar to the materials of the predetermined target region f₁. The vibration waves of the predetermined frequencies λᵣ₁ and λᵣ₂ respectively generated from the first vibrating region t₁ and the second vibrating region t₂ may be collected by the ultrasound probe 100.

The frequencies λᵣ₁ and λᵣ₂ of the vibration waves respectively generated from the materials of the first vibrating region t₁ and the second vibrating region t₂ may be influenced by the frequency λᵣ of the vibration waves generated from the materials of the predetermined target region f₁. The frequencies λᵣ₁ and λᵣ₂ of the vibration waves respectively generated from the materials of the first vibrating region t₁ and the second vibrating region t₂ may be substantially identical to or different from the frequency λᵣ of the vibration waves generated from the materials of the predetermined target region f₁.

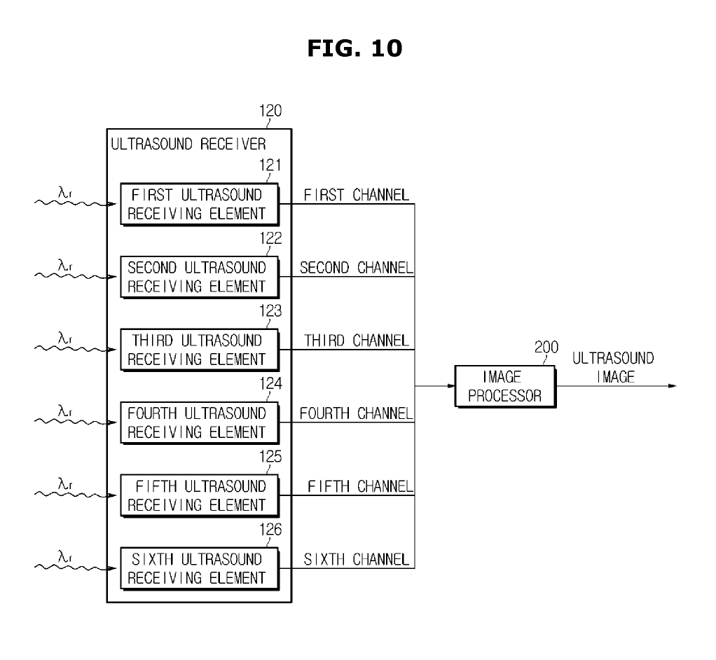

Hereinafter, the ultrasound receiver 120 will be described. FIG. 10 is a block diagram illustrating a configuration of the ultrasound receiver 120 according to an exemplary embodiment. As shown in FIGS. 3 and 10, the ultrasound probe 100 may include the ultrasound receiver 120 to receive vibration waves transferred from the object ob. The ultrasound receiver 120 may receive vibration waves of a predetermined frequency λᵣ, convert the vibration waves into electrical signals corresponding to the vibration waves, and output the electrical signals. The electrical signals may be transferred to the image processor 200. The ultrasound receiver 120 may output a plurality of electrical signals corresponding to a plurality of irradiation directions of ultrasonic waves. As described above with reference to FIGS. 6 to 8, when the ultrasound generator 110 irradiates ultrasonic waves to the target region f₁ of the object ob in a plurality of directions, the ultrasound receiver 120 may receive a plurality of vibration waves according to the irradiation of the ultrasonic waves in the plurality of directions. Herein, the ultrasonic waves irradiated by the ultrasound generator 110 may be ultrasonic waves of different frequencies that intersect each other at or around the target region f₁ inside the object ob, as described above. The ultrasound receiver 120 may output a plurality of ultrasound signals corresponding to the plurality of vibration waves. The plurality of ultrasound signals may be electrical signals of a plurality of channels, for example, first to sixth channels, as shown in FIG. 10.

According to an exemplary embodiment, the ultrasound receiver 120 may include a plurality of ultrasound receiving elements, e.g., a first ultrasound receiving element 121, a second ultrasound receiving element 122, a third ultrasound receiving element 123, a fourth ultrasound receiving element 124, a fifth ultrasound receiving element 125, and a sixth ultrasound receiving element 126, as shown in FIG. 10. The ultrasound receiving elements 121 to 126 may receive predetermined waves, and convert the received waves into electrical signals. More specifically, the ultrasound receiving elements 121 to 126 may receive vibration waves of a predetermined frequency λᵣ radiated from the target region f₁ or the vibrating regions t₁ and t₂, convert the received vibration waves into electrical signals, and output the electrical signals.

In detail, if the ultrasound receiving elements 121 to 126 receive vibration waves of a predetermined frequency generated from the target region f₁ or the vibrating regions t₁ and t₂, the ultrasound receiving elements 121 to 126 may vibrate at a predetermined frequency corresponding to the frequency of the received vibration waves. The vibrating ultrasound receiving elements 121 to 126 may output alternating current of the vibration frequency of the ultrasound receiving elements 121 to 126. Accordingly, the ultrasound receiver 120 may convert the received vibration waves into predetermined electrical signals.

To convert the received vibration waves into electrical signals, the ultrasound receiving elements 121 to 126 may be ultrasonic transducers. The ultrasonic transducer may be a device for converting one form of energy into another form of energy. For example, the ultrasonic transducer may convert electrical signals into sound energy or convert sound energy into electrical signals. Ultrasonic transducers that are used as the ultrasound receiving elements 121 to 126 may be piezoelectric ultrasonic transducers using a piezoelectric effect of a piezoelectric material, magnetostrictive ultrasonic transducers that convert wave energy into electricity energy using a magnetostrictive effect of a magnetic material, or capacitive micromachined ultrasonic transducers (CMUTs) that transmit and receive ultrasonic waves using vibration of several hundreds or thousands of micromachined thin films. However, the ultrasound generating elements 121 to 126 may be any other type ultrasonic transducers capable of generating ultrasonic waves according to electrical signals or generating electrical signals according to ultrasonic waves.

As shown in FIG. 10, if the ultrasound receiver 120 includes the plurality of ultrasound receiving elements 121 to 126, the ultrasound receiver 120 may output electrical signals of a plurality of channels, e.g., a first to a sixth channels, since the respective ultrasound receiving elements 121 to 126 output electrical signals.

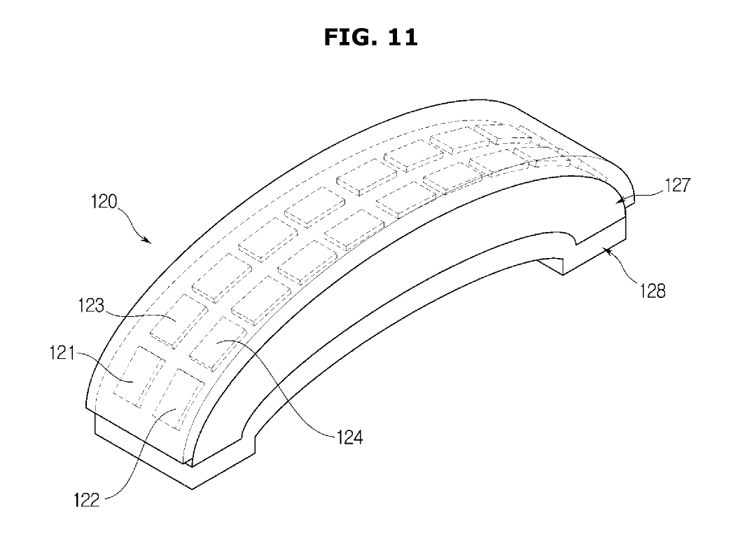

FIG. 11 is a perspective view of the ultrasound receiver 120 according to an exemplary embodiment. According to an exemplary embodiment, as shown in FIG. 11, a plurality of ultrasound receiving elements 121 to 124 may be arranged on a frame 127 of the ultrasound receiver 120. In this case, the ultrasound receiving elements 121 to 124 may be arranged on the frame 127 according to a predetermined pattern. For example, the ultrasound receiving elements 121 to 124 may be arranged in at least one row on the frame 127. For example, as shown in FIG. 11, the ultrasound receiving elements 121 to 124 are arranged in two rows on the frame 127.

The frame 127 may include a resting groove or a protrusion formed on a side on which the ultrasound receiving elements 121 to 124 are arranged, so that the ultrasound receiving elements 121 to 124 can be stably arranged and fixed according to a predetermined pattern. The ultrasound receiving elements 121 to 124 may be arranged on a groove or protrusion of a predetermined pattern.

To stably fix the ultrasound receiving elements 121 to 124 on the frame 127, a predetermined adhesive, for example, epoxy resin adhesive may be used. The predetermined adhesive may be applied between the ultrasound receiving elements 121 to 124 and the frame 127 to bond the ultrasound receiving elements 121 to 124 to the frame 127, thereby fixing the ultrasound receiving elements 121 to 124. However, for purpose of bonding and fixing the ultrasound receiving elements 121 to 124 to the frame 127, any other kind of coupling, fixing, and bonding means may be used.

On a side opposite to the side on which the ultrasound receiving elements 121 to 124 are arranged may be formed a substrate 128 to control current that is applied to the individual ultrasound receiving elements 121 to 124. On the substrate 128 may be formed various circuitry to control the ultrasound receiving elements 121 to 124 or to control communication of the ultrasound receiving elements 121 to 124 with an external main body of the ultrasound probe 100.

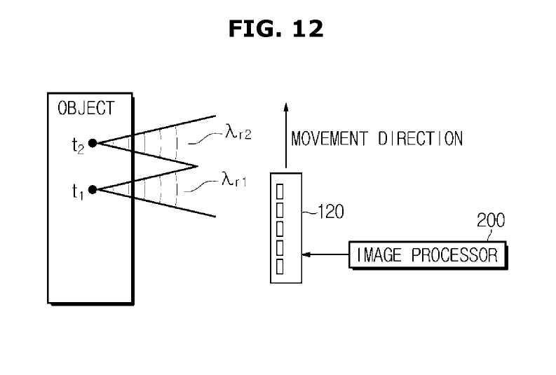

FIG. 12 is a view for describing the ultrasound receiver 120 according to an exemplary embodiment. As shown in FIG. 12, the ultrasound receiver 120 may move in a predetermined direction to receive a plurality of vibration waves that are radiated from a plurality of vibrating regions t₁ and t₂ of an object ob.

As described above with reference to FIG. 9, materials of the target region f₁ may vibrate according to the results of interference of ultrasonic waves of different frequencies λ₁₁ and λ₁₂ to generate vibration waves that are transferred to vibrating regions t₁ and t₂ around the target region f₁. Then, the vibrating regions t₁ and t₂ around the target region f₁ may vibrate according to the vibration waves to radiate vibration waves of predetermined frequencies λᵣ₁ and λᵣ₂. Then, the vibration waves generated from the target region f₁ move through the inside of the object ob at a predetermined velocity, and arrive at a vibrating region (for example, the first vibrating region t₁) that is closer to the target region f₁. Then, the vibration waves may arrive at a vibrating region (for example, the second vibrating region t₂) that is distanced away from the target region f₁ than the first vibrating region t₁, later than the time at which the vibration waves have arrived at the first vibrating region t₁. Accordingly, the first and second vibrating regions t₁ and t₂ may generate vibration waves at different times. In other words, vibration waves may be sequentially generated from a region (for example, the first vibrating region t₁) that is closer to the target region f₁ and a region (for example, the second vibrating region t₂) that is distanced away from the target region f₁. If vibration waves are generated in this way, the ultrasound receiver 120 may move in a predetermined direction to receive the vibration waves sequentially generated from the vibrating regions t₁ and t₂, as shown in FIG. 12. In this case, the movement direction of the ultrasound receiver 120 may be set according to a movement direction of vibration waves generated from the target region f₁. Due to movement of the ultrasound receiver 120, the ultrasound receiver 120 may receive a plurality of vibration waves of predetermined frequencies λᵣ₁ and λᵣ₂ that are output from all the vibrating regions t₁ and t₂. Accordingly, all regions inside the object ob may be scanned.

According to an exemplary embodiment, the ultrasound receiver 120 may move to receive vibration waves, and transfer the received vibration waves to the image processor 200 in real time so that the image processor 200 can generate a predetermined ultrasound image. According to another exemplary embodiment, the ultrasound receiver 120 may move to receive vibration waves, store the received vibration waves in a storage, and transfer the vibration waves to the image processor 200 at regular time intervals or after vibration waves are completely received so that the image processor 200 can produce a predetermined ultrasound image.

However, instead of moving the ultrasound receiver 120, the ultrasound receiver 120 may be fixed at a predetermined location so that the ultrasonic receiver 120 can receive a plurality of vibration waves of predetermined frequencies λᵣ₁ and λᵣ₂ radiated from the plurality of vibrating regions t₁ and t₂ of the object ob.

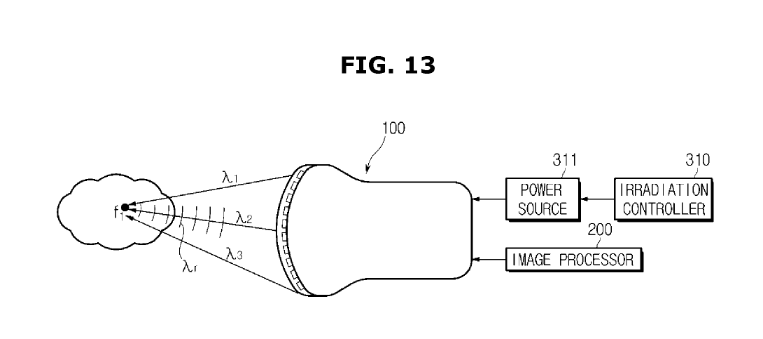

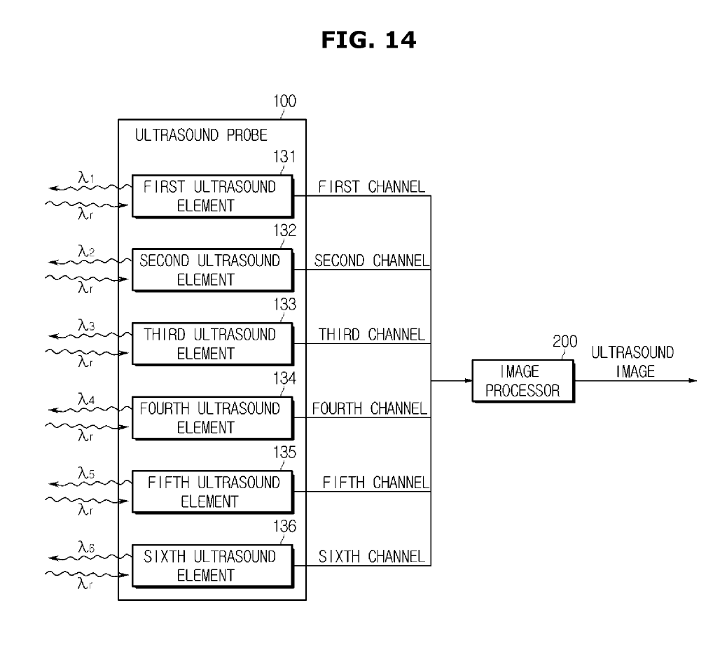

Hereinafter, an ultrasound probe according to other exemplary embodiments will be described. FIGS. 13 and 14 are views for describing an ultrasound probe according to other exemplary embodiments. As shown in FIGS. 13 and 14, an ultrasound probe 100 may include a plurality of ultrasound elements, e.g., a first ultrasound element 131, a second ultrasound element 132, a third ultrasound element 133, a fourth ultrasound element 134, a fifth ultrasound element 135, and a sixth ultrasound element 136 to generate and receive ultrasonic waves.

The plurality of ultrasound elements 131 to 136 may generate ultrasonic waves of a plurality of different frequencies, for example, first to third frequencies λ₁ to λ₃ according to current that is applied to the ultrasound elements 131 to 136 based on power received from an external power source 311, irradiate the ultrasonic waves of the different frequencies to a target region f₁ of an object ob, receive vibration waves of a predetermined frequency λᵣ generated from the object ob, and convert the received vibration waves into electrical signals.

In detail, the plurality of ultrasound elements 131 to 136 may vibrate at a frequency of alternating current that is applied from the external power source 311. The plurality of ultrasound elements 131 to 136 may vibrate to generate ultrasonic waves of frequencies λ₁ to λ₃ corresponding to vibration frequencies thereof. In this case, the plurality of ultrasound elements 131 to 136 may be grouped into a plurality of groups, and different frequencies of alternating current may be applied to the respective groups to generate ultrasonic waves of different frequencies λ₁ to λ₃. However, the ultrasound elements 131 to 136 may generate ultrasonic waves of different frequencies λ₁ to λ₆, respectively, as shown in FIG. 14. Hereinafter, for illustrative purposes, description is made with respect to the case where the plurality of ultrasound elements 131 to 136 may be grouped into a plurality of groups to generate different frequencies λ₁ to λ₃.

Whether to apply current to the plurality of ultrasound elements 131 to 136, or a frequency of current that is to be applied to the plurality of ultrasound elements 131 to 136 may be controlled by an irradiation controller 310.

As described above with reference to FIGS. 6 to 8, the ultrasound probe 100 can apply ultrasonic waves to the target region f₁ of the object ob in a plurality of directions. In this case, the ultrasound probe 100 may generate ultrasonic waves of different frequencies intersecting each other at the target region f₁ inside the object ob in each direction, and irradiate the ultrasonic waves to the target region f₁ inside the object ob.

The ultrasonic waves of the different frequencies λ₁ to λ₃ generated by the plurality of ultrasonic elements 131 to 136 may intersect each other at or around the target region f₁ of the object ob, and vibrate materials of the target region f₁ according to an interference frequency due to the intersection of the ultrasonic waves of the different frequencies λ₁ to λ₃. As a result, the materials of the target region f₁ or the materials of the vibrating regions t₁ and t₂ around the target region f₁ vibrate to produce predetermined vibration waves.

Accordingly, the plurality of ultrasound elements 131 to 136 that have irradiated the ultrasonic waves of the different frequencies λ₁ to λ₃ may receive vibration waves transferred from the target region f₁ or from the vibrating regions t₁ and t₂ around the target region f₁. The plurality of ultrasound elements 131 to 136 may vibrate according to the received frequency to output alternating current of the vibration frequency.

The ultrasound probe 100 may convert the received vibration waves into predetermined electrical signals. Since electrical signals are output from the respective ultrasound elements 131 to 136, the ultrasound probe 130 may output electrical signals of a plurality of channels. The electrical signals of the plurality of channels may be transferred to the image processor 200.

As shown in FIGS. 6 to 8, when ultrasonic waves are irradiated to the target region f₁ of the object ob in a plurality of directions, the ultrasound probe 130 may receive a plurality of vibration waves according to the irradiation of the ultrasonic waves in the plurality of directions. As a result, the ultrasound probe 100 may output a plurality of electrical signals of a plurality of channels.

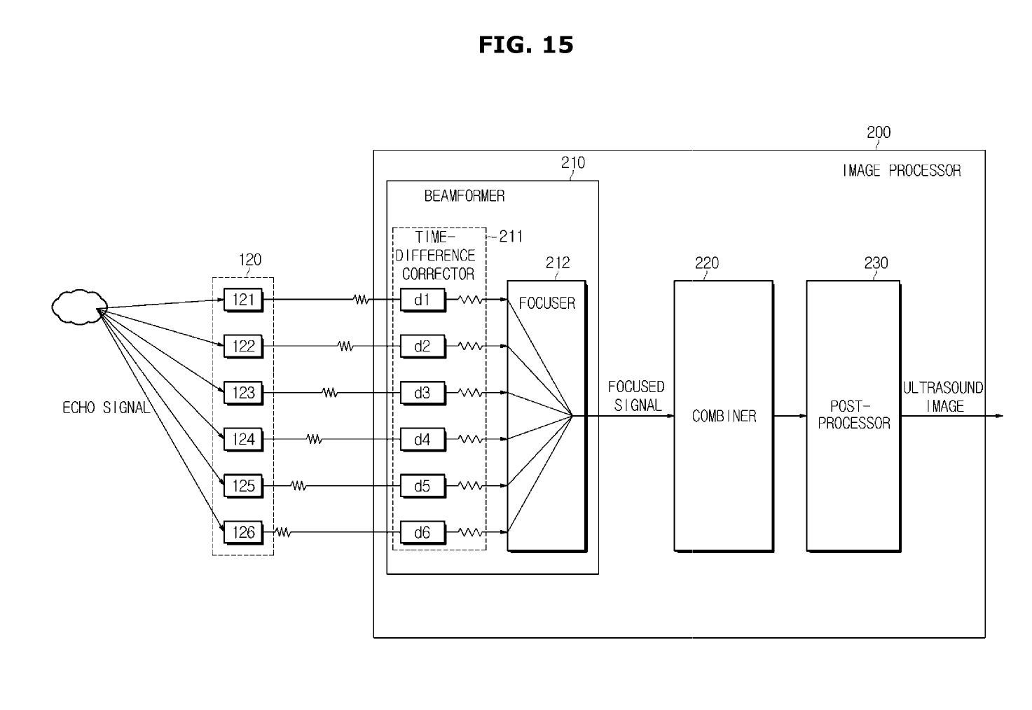

FIG. 15 is a block diagram illustrating a configuration of the image processor 200 according to an exemplary embodiment. As described above, if the ultrasound probe 100 or the ultrasound receiver 120 of the ultrasound probe 100 outputs electrical signals of at least one channel, the image processor 200 may focus the electrical signals of the at least one channel to produce an ultrasound image.

The image processor 200 may receive electrical signals of a plurality of channels transferred from the ultrasound receiving elements 121 to 126, as shown in FIG. 10, or from the ultrasound elements 131 to 136, as shown in FIG. 14, focus the electrical signals of the plurality of channels to form beamforming on the ultrasound signals of the plurality of channels to estimate the magnitudes of reflected waves in a specific space, produce an ultrasound image using the beamformed ultrasound signals, and output the ultrasound image. As shown in FIG. 15, the image processor 200 may include a beamformer 210, a combiner 220, and a post-processor 230.

The beamformer 210 may perform beamforming of a plurality of channels. The beamformer 210 may include a time-difference corrector 211 and a focuser 212.

The time-difference corrector 211 may correct time differences between ultrasound signals output from the ultrasound receiving elements 121 to 126 or the ultrasound elements 131 to 136.

As described above, the ultrasound receiving elements 121 to 126 or the ultrasound elements 131 to 136 may receive vibration waves from the target region f₁ or the vibrating regions t₁ and t₂. Since the ultrasound receiving elements 121 to 126 or the ultrasound elements 131 to 136, for example, transducers are spaced by different distances away from the target region f₁, the ultrasound receiving elements 121 to 126 or the ultrasound elements 131 to 136 may receive vibration waves transferred from the target region f₁ or from the vibrating regions t₁ and t₂, at different times, respectively. Therefore, electrical signals of individual channels that are output from the ultrasound receiving elements 121 to 126 or the ultrasound elements 131 to 136 may have predetermined time differences therebetween. The time-difference corrector 211 may correct time differences between the electrical signals of the individual channels so that the focuser 212 can focus electrical signals acquired according to substantially the same vibration waves.

To correct time differences between ultrasound signals, for example, as shown in FIG. 15, the time-difference corrector 211 including time-difference correctors d1, d2, d3, d4, d5, and d6 may delay transmission of ultrasound signals of predetermined channels output from the ultrasound receiving elements 121 to 126 by predetermined time periods so that the ultrasound signals of the plurality of channels can be transferred to the focuser 212 at substantially the same time.

The focuser 212 may focus, as shown in FIG. 15, the ultrasound signals of the plurality of channels subject to time-difference correction by the time-difference corrector 211, and output the focused signal.

According to an exemplary embodiment, the focuser 212 may focus the ultrasound signals of the plurality of channels after allocating a predetermined weight (for example, a beamforming coefficient) to each ultrasound signal to enhance or attenuate an ultrasound signal at a predetermined location rather than the other ultrasound signals. Accordingly, it is possible to produce a user's desired ultrasound image.

The focuser 212 may focus ultrasound signals using pre-defined beamforming coefficients without considering the ultrasound signals, in case of data-independent beamforming. Also, the focuser 212 may acquire appropriate beamforming coefficients based on received ultrasound signals, and focus the ultrasound signals using the acquired beamforming coefficients, in case of data-dependent beamforming.

The beamforming process that is performed by the time-difference corrector 211 and the focuser 212 can be expressed by Equation (4), below.

z[n]=Σₘ₌₀^{M-1}wₘ[n]xₘ[n−Δₘ[n]], (4)

where n is an index for a location of a target region such as a depth of the target region, m is an index for each channel, and wₘ is a weight (for example, a beamforming coefficient) allocated to an ultrasound signal of an m-th channel. Δₘ is a time-difference correction value. The time-difference correction value is a value that the time-difference corrector 211 uses to delay a transmission time of an ultrasound signal. According to Equation (4), the focuser 212 may focus electrical signals of individual channels subject to time correction, and output a focused signal. The focused signal may be used as an ultrasound image.

According to an exemplary embodiment, the focused signal that is output from the beamformer 210 may be transferred to the combiner 220 as shown in FIG. 15.

As described above with reference to FIGS. 6, 7, and 8, the ultrasound probe 100 may irradiate ultrasonic waves to the target region f₁ in a plurality of directions. In this case, the ultrasonic waves that are irradiated to the target region f₁ may be ultrasonic waves of different frequencies intersecting each other at the target region f₁. The ultrasound probe 100 or the ultrasound receiver 120 may receive a plurality of vibration waves corresponding to irradiation directions of the plurality of ultrasonic waves, and output a plurality of electrical signals of a plurality of channels. The plurality of electrical signals of the plurality of channels may be focused by the beamformer 210. As a result, a plurality of ultrasound images may be acquired.

The combiner 220 may combine a plurality of focused signals, that is, a plurality of ultrasound images to produce a combined signal, that is, a combined ultrasound image.

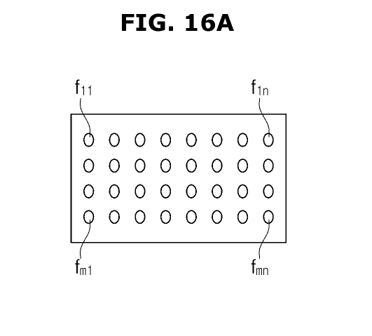

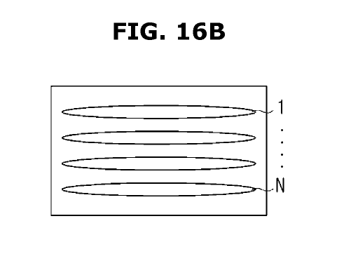

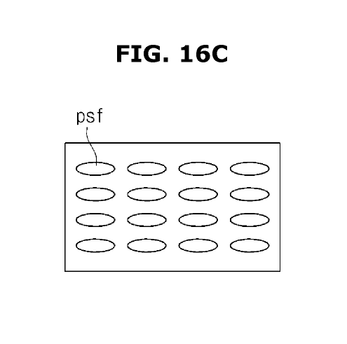

FIGS. 16 to 18 are views for describing a method of combining a plurality of images according to exemplary embodiments. FIG. 16A illustrates a plurality of materials included in an object ob. In FIG. 16A, each material is, for convenience of description, shown in the form of a circle arranged in rows and columns. The individual materials are located in a plurality of target regions or vibrating regions f₁₁ to fₘₙ. The materials of a plurality of target regions or vibrating regions f₁₁ to f₁ₙ shown in a top row may be materials of a plurality of target regions or vibrating regions that are closest to the ultrasound probe 100, and materials of a plurality of target regions or vibrating regions fₘ₁ to fₘₙ shown in a bottom row may be materials of a plurality of target regions or vibrating regions that are most distant from the ultrasound probe 100.

FIG. 16B illustrates locations of vibration waves that are generated from a plurality of target regions or vibrating regions f₁₁ to fₘₙ according to ultrasound irradiation by the ultrasound probe 100. As described above with reference to FIGS. 6, 7, and 8, if ultrasound waves of different frequencies are irradiated to a target region, the target region or a vibrating region around the target region may vibrate due to an interference frequency caused by the different frequencies of the irradiated ultrasound waves, to generate vibration waves. Vibration waves that are generated from the plurality of target regions or vibrating regions f₁₁ to fₘₙ are shown in FIG. 16B, and 1 to N are indexes for depths. If the ultrasound probe 100 cannot irradiate ultrasonic waves to target regions of different depths, for example, the target region f₁₁ in the top row (i.e., depth index=1) and the target region fₘ₁ in the bottom row (i.e., depth index=N), at substantially the same time, the ultrasound probe 100 may need to irradiate ultrasonic waves N times to acquire vibration waves of 1 to N depths.

FIG. 16C shows focused signals (that is, images) that are transferred to the combiner 220. If the ultrasound probe 100 receives vibration waves, and the beamformer 210 focuses electrical signals of one or more channels corresponding to the received vibration waves, ultrasound image signals as shown in FIG. 16C may be acquired.

Each ellipse shown in FIG. 16C represents an ultrasound image signal that is acquired when each material shown in FIG. 16A is imaged, and may represent a point spread function (PSF) for a material of a predetermined target region or a predetermined vibrating region f₁₁ to fₘₙ. The PSF is a function that expresses a relationship between an ideal image and acquired radio frequency (RF) image data. When a predetermined object is imaged using an imaging apparatus, acquired image signals may be different from an ideal image due to the technical properties or physical properties (for example, scratches of ultrasound receiving elements) of the imaging apparatus. A function that expresses such a difference is a PSF.

As such, a material shape acquired by an ultrasonic imaging apparatus may be different from an original material shape. For example, a material which is in the shape of a circle as shown in FIG. 16A may be acquired in the shape of an ellipse as shown in FIG. 16C by the ultrasonic imaging apparatus.

The combiner 220 may combine a plurality of ultrasound images to acquire the shape of an original material, that is, a combined ultrasound image that is substantially identical to or similar to an ideal image.

According to an exemplary embodiment, the combiner 220 may combine a plurality of images in a plurality of directions, produced based on a plurality of vibration waves generated according to irradiation of ultrasonic waves in the plurality of directions, thereby acquiring a combined ultrasound image.

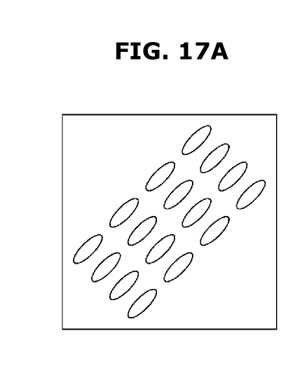

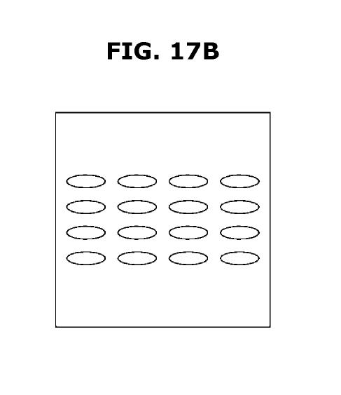

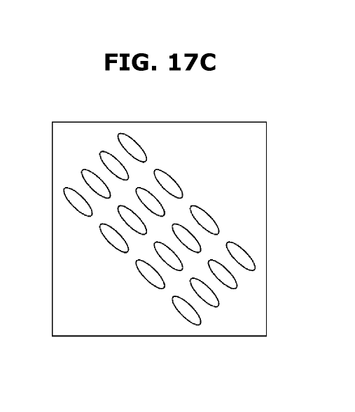

FIGS. 17A to 17C show a plurality of images acquired by irradiating ultrasonic waves to a target region of an object in a plurality of directions according to an exemplary embodiment. The ultrasonic waves irradiated in the respective directions means a plurality of ultrasonic waves of different frequencies.

FIG. 17A shows an image acquired by irradiating ultrasonic waves at a predetermined angle, e.g., about 45 degrees with respect to the target regions of FIG. 16A and receiving radiated vibration waves. FIG. 17B shows an image acquired by irradiating ultrasonic waves at about 90 degrees with respect to the target regions and receiving radiated vibration waves. FIG. 17C shows an image acquired by irradiating ultrasonic waves at about 135 degrees with respect to the target regions and receiving radiated vibration waves.

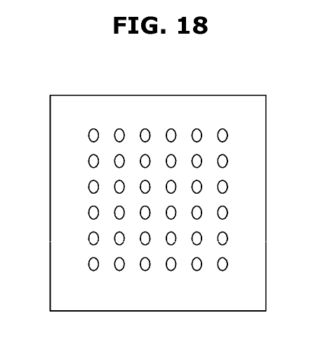

The combiner 220 may combine a plurality of images as shown in FIGS. 17A, 17B, and 17C, acquired according to irradiation of ultrasonic waves in a plurality of directions, and acquire a combined ultrasound image as shown in FIG. 18. Through the combination, the magnitudes of vibration waves for all the target regions or vibrating regions f₁₁ to fₘₙ can be extracted. Also, a combined ultrasound image that is substantially identical or similar to the shapes of original materials, that is, an ideal image can be acquired.

The combined ultrasound image may be stored in a predetermined storage unit, such as, for example, a buffer, a random access memory (RAM), a magnetic disk, a semiconductor memory, or an optical memory, which can store electrical signals temporarily or non-temporarily. The combined ultrasound image may be transferred to and displayed on a predetermined display, for example, a monitor. The acquired ultrasound image may be transferred to the post-processor 230.

The post-processor 230 may perform predetermined image processing on the ultrasound image combined by the combiner 220. For example, the post-processor 230 may correct at least one from among the luminosity, brightness, contrast, and sharpness of an entire or a part of the ultrasound image. In this case, the post-processor 230 may correct the ultrasound image according to an instruction or a command from a user or according to a pre-defined setting. Also, if a plurality of ultrasound images are output from the combiner 220, the post-processing 230 may combine the plurality of ultrasound images to produce a three-dimensional (3D) stereo ultrasound image. The combined ultrasound image processed by the post-processor 230 may also be stored in the predetermined storage unit, and displayed on a display device.

Hereinafter, a control method of the ultrasonic imaging apparatus will be described.

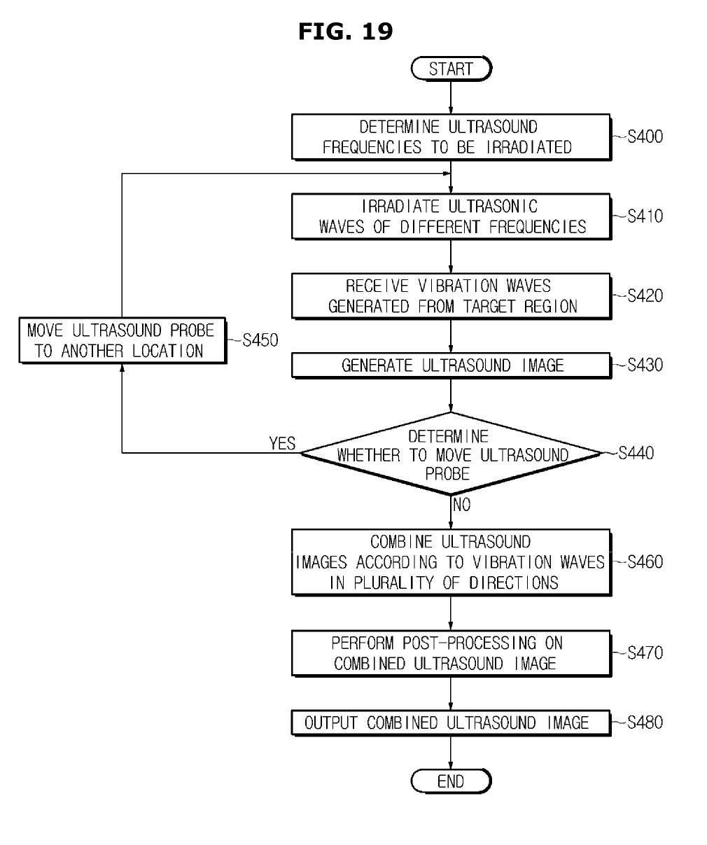

FIG. 19 is a flowchart illustrating a control method of an ultrasonic imaging apparatus, according to an exemplary embodiment. As shown in FIG. 19, according to an exemplary embodiment of a control method of an ultrasonic imaging apparatus, a plurality of ultrasound frequencies to be irradiated may be determined in operation S400. The determined ultrasound frequencies mean frequencies of ultrasonic waves that are to be interfered with each other. The frequencies of the ultrasonic waves that are to be interfered with each other may be different from each other. Hereinafter, for illustrative purposes, description is made with respect to the case where a plurality of ultrasound waves have different frequencies.

Next, a plurality of ultrasonic waves of different frequencies according to the determined ultrasound frequencies may be irradiated to a target region in a predetermined direction, in operation S410. The plurality of ultrasonic waves of the different frequencies may be irradiated by an ultrasound probe. The ultrasound probe may be movable. The plurality of ultrasonic waves of the different frequencies irradiated to the target region may intersect and interfere with each other so that a predetermined interference frequency applies vibration to the target region. Materials of the target region may radiate vibration waves according to the applied vibration. In this case, the radiated vibration waves may be transferred to materials around the materials of the target region, and the materials that have received the vibration waves radiated from the target region may generate vibration waves.

The vibration waves generated from the materials of the target region and the materials around the target region may be received by the ultrasound probe in operation S420. The ultrasound probe may convert the received vibration waves into a plurality of electrical signals of a plurality of channels.

The electrical signals of the plurality of channels may be applied with time-difference correction, and then be focused. Accordingly, an ultrasound image corresponding to the vibration waves may be acquired in operation S430.

According to determination on whether to irradiate ultrasonic waves in another direction, whether to move the ultrasound probe may be determined in operation S440. If ultrasonic waves need to be irradiated in another direction, the ultrasound probe may be moved in the corresponding direction, in operation S450. After the ultrasound probe is moved, a plurality of ultrasonic waves of different frequencies may be irradiated at the moved location of the ultrasound probe, and an ultrasound image at the moved location may be acquired in substantially the same method as described above.

Operations S410 to S450 may be repeatedly performed several times to acquire ultrasound images according to a plurality of vibration waves acquired by irradiation of ultrasonic waves at a plurality of locations.

If a plurality of ultrasound images are acquired according to a plurality of vibration waves, the plurality of ultrasound images may be combined, in operation S460.

Predetermined post-processing may be performed on the combined ultrasound image, in operation S470.

After the predetermined post-processing, the combined ultrasound image may be displayed through a display, for example, a monitor, in operation S480.

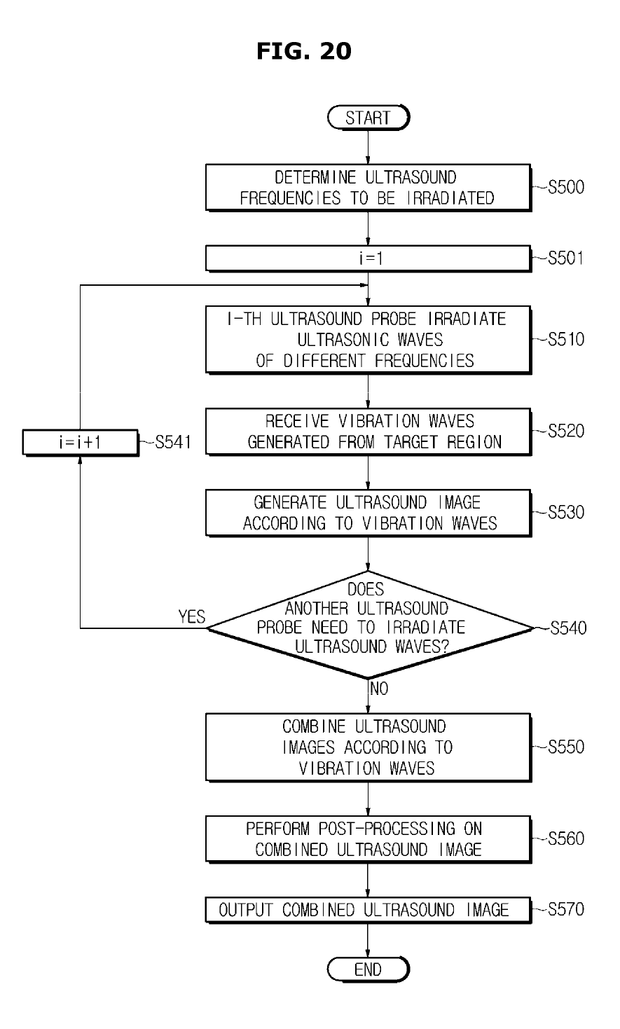

FIG. 20 is a flowchart illustrating a control method of an ultrasonic imaging apparatus, according to another exemplary embodiment.

The control method illustrated in FIG. 20 may be performed by an ultrasonic imaging apparatus including a plurality of ultrasound probes.

First, a plurality of ultrasound frequencies that are to be irradiated may be determined, in operation S500. The determined ultrasound frequencies mean frequencies of ultrasonic waves that are to be interfered with each other at a predetermined target region. The frequencies of the ultrasonic waves that are to be interfered with each other may be different from each other. Hereinafter, for illustrative purposes, description is made with respect to the case where a plurality of ultrasound waves have different frequencies.

According to the decision in operation S500, a plurality of ultrasonic waves of different frequencies may be irradiated to a target region. In this case, the plurality of ultrasonic waves of the different frequencies may be irradiated by a first ultrasound probe of the plurality of ultrasound probes, in operations S501 and S510.

Then, vibration waves generated from materials of the target region or from materials around the target region according to the interference of the plurality of ultrasonic waves of the different frequencies may be received. In this case, the vibration waves may be received by the first ultrasound probe that has irradiated the ultrasonic waves, or by another ultrasound probe, in operation S520.

The vibration waves received by the first ultrasound probe, or by the another ultrasound probe, may be converted into electrical signals of a plurality of channels, and the electrical signals of the plurality of channels may be applied with time-difference correction and then focused. As a result, an ultrasound image corresponding to the vibration waves received by the first ultrasound probe, or by the another ultrasound probe, may be acquired, in operation S530.

Next, whether another ultrasound probe other than the first ultrasound probe needs to irradiate ultrasound waves may be determined. That is, whether ultrasonic waves need to be irradiated in another direction may be determined, in operation S540.

If ultrasound waves need to be irradiated in another direction, operations S510 to S530 may be repeatedly performed so that an ultrasound image is acquired based on vibration waves generated according to irradiation of ultrasonic waves in the another direction, in operation S541. Accordingly, a plurality of ultrasound images according to a plurality of vibration waves may be acquired.

The plurality of ultrasound images according to the plurality of vibration waves may be combined, in operation S550

The combined ultrasound image may be applied with predetermined post processing, in operation S560. The combined ultrasound image may be output through a display, for example, a monitor, in operation S570.

In the ultrasonic imaging apparatus and the control method thereof according to exemplary embodiments, since signals used for generating an ultrasound image can be quickly collected, an ultrasound image can be acquired at high speed.

Also, it is possible to quickly acquire higher resolution ultrasound images.

In addition, it is possible to acquire an ultrasound image based on vibroacoustography without using a hydrophone while reducing a time consumed to collect ultrasonic waves.

Also, it is possible to produce a higher resolution image by improving reception focusing at lower resolution to prevent resolution deterioration of acquired images in vibroacoustography.

The foregoing exemplary embodiments and advantages are merely exemplary and are not to be construed as limiting. The present teaching can be readily applied to other types of apparatuses. The description of the exemplary embodiments is intended to be illustrative, and not to limit the scope of the claims, and many alternatives, modifications, and variations will be apparent to those skilled in the art.

What is claimed is:

1. An ultrasonic imaging apparatus comprising:
an ultrasound probe configured to transmit ultrasonic waves to a target region of an object in a plurality of directions, and to receive vibration waves generated from the object; and
an image processor configured to generate image signals in the plurality of directions based on the vibration waves generated according to transmission of the ultrasonic waves in the plurality of directions, and to combine the image signals in the plurality of directions,
wherein the ultrasound probe comprises ultrasound elements configured to respectively generate ultrasonic waves of different frequencies, the ultrasonic waves intersecting each other in the target region of the object.

2. The ultrasonic imaging apparatus according to claim 1, wherein the ultrasound probe is configured to be movable.

3. The ultrasonic imaging apparatus according to claim 1, wherein the ultrasound elements are configured to receive the vibration waves, and convert the received vibration waves to generate an echo signal.

4. The ultrasonic imaging apparatus according to claim 1, wherein the ultrasound probe further comprises a support frame on which the ultrasound elements are arranged in a row.

5. The ultrasonic imaging apparatus according to claim 3, wherein the image processor comprises:
a focuser configured to focus the echo signal, and to acquire the image signals in the plurality of directions; and
a combiner configured to combine the image signals in the plurality of directions, focused by the focuser, and to generate a combined image.

6. The ultrasonic imaging apparatus according to claim 1, wherein the ultrasound elements comprise:
an ultrasound generator configured to generate and irradiate the ultrasonic waves of the different frequencies; and
an ultrasound receiver configured to receive the vibration waves that are generated from the target region according to interference waves generated by the ultrasonic waves of the different frequencies intersecting each other in the target region.

7. The ultrasonic imaging apparatus according to claim 6, wherein the ultrasound generator is configured to be movable.

8. The ultrasonic imaging apparatus according to claim 6, wherein the ultrasound receiver comprises a receiving element configured to receive the vibration waves, and to convert the vibration waves to generate an echo signal.

9. The ultrasonic imaging apparatus according to claim 6, wherein the ultrasound receiver comprises a hydrophone configured to receive waves.

10. The ultrasonic imaging apparatus according to claim 1, wherein the ultrasound probe is configured to transmit the ultrasonic waves of the different frequencies using the target region as a focal point.

11. The ultrasonic imaging apparatus according to claim 1, wherein an angle between first and second directions among the plurality of directions is substantially a right angle.

12. A control method of an ultrasonic imaging apparatus, the control method comprising:
transmitting ultrasonic waves of different frequencies in a plurality of directions to a target region of an object;
receiving vibration waves corresponding to the plurality of directions, generated from the target region according to interference of the ultrasonic waves of the different frequencies;
acquiring image signals in the plurality of directions based on the vibration waves; and
combining the image signals in the plurality of directions to generate a combined image.

13. The control method according to claim 12, wherein the acquiring the image signals comprises sequentially transmitting by ultrasound probes arranged at a plurality of locations, the ultrasonic waves of the different frequencies to the target region to acquire the image signals in the plurality of directions.

14. The control method according to claim 12, wherein the acquiring the image signals comprises moving an ultrasound probe to a plurality of locations to transmit the ultrasonic waves of the different frequencies at the plurality of locations to acquire the image signals in the plurality of directions.

15. The control method according to claim 12, wherein the acquiring the image signals comprises converting, by a receiving element of an ultrasound probe, the vibration waves to generate an echo signal.

16. The control method according to claim 15, wherein the receiving element is configured to be arranged in a row within the ultrasound probe.

17. The control method according to claim 15, wherein the acquiring the image signals further comprises:
converting the vibration waves into a plurality of echo signals;
correcting time differences between the plurality of echo signals; and
focusing the plurality of echo signals of which the time differences are corrected to acquire the image signals in the plurality of directions based on the vibration waves.

18. The control method according to claim 12, wherein the receiving the vibration waves comprises receiving, by a hydrophone, the vibration waves.

19. The control method according to claim 12, wherein the transmitting the ultrasonic waves comprises transmitting the ultrasonic waves of the different frequencies using the target region of the object as a focal point.

20. An ultrasonic imaging apparatus comprising:
an ultrasound probe configured to transmit ultrasonic waves of different frequencies in a plurality of directions, the ultrasonic waves intersecting each other in at a target region inside an object, and receive vibration waves generated from the object according to interference of the ultrasonic waves of the different frequencies; and
an image processor configured to generate images of the object in the plurality of directions based on the vibration waves generated according to transmission of the ultrasonic waves of the different frequencies in the plurality of directions, and combine images of the object in the plurality of directions to generate a combined image.
